(19) United States
(12) Patent Application Publication
(10) Pub. No.: US 2008/0306578 A1
(43) Pub. Date: Dec. 11, 2008

(54) Attachment of Tubing in a Cardiac Lead

(76) Inventors: Sara Hallander, Stockholm (SE); Susanne Nilsson, Huddinge (SE); Kenneth Dowling, Bro (SE); Eva Micski, Huddinge (SE); Tom Eriksson, Uppsala (SE)

Correspondence Address: SCHIFF HARDIN, LLP;PATENT DEPARTMENT, 6600 SEARS TOWER, CHICAGO, IL, 60606-6473, US

(21) Appl. No.: 11/667,643

(22) PCT Filed: Nov. 24, 2004
(86) PCT No.: PCT/SE04/01738

§ 371 Date: Nov. 16, 2007

(51) Int. Cl. A61N 1/05; B32B 37/06

(52) U.S. Cl. 607/126; 156/73.1

(57) ABSTRACT

In a method for fixing tubing of a cardiac lead to a termination element, such as an electrode or a pin connector and in a cardiac lead formed according to such a method, an exterior of the termination element is provided with a thermoplastic fixation element, and thermoplastic tubing is provided with an end portion that coaxially surrounds a portion of the fixation element. A section of the end portion of the thermoplastic tubing is fused to the thermoplastic fixation element, so that the thermoplastic tubing is fixed to the termination element.

### BACKGROUND OF THE INVENTION

1. Field of the Invention

The present invention relates to a cardiac lead of the type having a thermoplastic tubing and an electrode or a pin connector, and to a method for fixing a thermoplastic cardiac lead tubing to a cardiac lead electrode or pin connector.

2. Description of the Prior Art

A cardiac lead may be a bipolar (or unipolar or multipolar) electrode lead used for providing stimulation of cardiac tissue, and/or for sensing heart signals, by means of a pulse generator or some other type of heart stimulation apparatus. The cardiac lead carries the stimulus from the pulse generator to the cardiac tissue, or relays intrinsic cardiac signals back to a sense amplifier of such pulse generator.

In a known cardiac lead, an electrically conducting coil interconnects a cardiac electrode (mounted at a distal end of the lead) and a connector (for connection to a cardiac stimulation device). The coil is surrounded by a tubing (for protection and electrical insulation of the coil) that is attached to the electrode and the connector, respectively, at the ends of the cardiac lead. The tubing material is commonly silicone, which is advantageously fixed to the electrode or connector by a silicone adhesive. Such adhesives are commonly used in medical applications.

A desire for improved tubing properties, such as increased resistance to abrasion and altered flexibility, has lead to the use of other tubing material, such as organic polymeric materials. However, due to their intrinsic properties such polymeric materials cannot be effectively fixed to the electrode or connector by silicone adhesives. Other adhesives which may render effective fixation are known. These are, however, not approved for medical use. As an alternative, the solvent method has been applied for joining the tubing to the electrode. Disadvantageously, joints obtainable by said method are difficult to make accurately and also difficult to inspect.

### SUMMARY OF THE INVENTION

An object of the present invention is to provide an alternative, improved cardiac lead, wherein a thermoplastic tubing is attached to an electrode or a pin connector.

Another object of the present invention is to provide an alternative and improved method for attaching a thermoplastic tubing to an electrode or a pin connector.

The invention is based on the insight that the intrinsic properties of thermoplastic tubing materials can be utilised for their attachment to electrical contact means, such as an electrode or a pin connector, without the use of glues or adhesives. Hence, improved strength and simple production processes are achieved. Furthermore, no separate adhesive is necessary, thereby reducing costs and avoiding regulatory procedures concerning materials for use in medical devices.

Thus, in accordance with the present invention a cardiac lead has a thermoplastic tubing and an electrode or a pin connector, wherein said electrode or a pin connector is provided with outer thermoplastic fixation means, an end portion of the tubing is coaxially provided around a portion of the fixation means, and a section of the end portion of the tubing is fused to the fixation means, such that the tubing is fixed to the electrode or pin connector.

The fusing of the tubing and the fixation means provides a “seamless” attachment between the tubing and the electrode or pin connector, conferring mechanical strength, as well as an insulating closure.

The fixation means is preferably provided in a recess in the electrode or pin connector. Such fixation means can easily be casted during production of the contact means and provides for a durable attachment. Although not necessary, for the ease of production and quality of the attachment said thermoplastic fixation means may extend essentially around the perimeter of the electrical contact means.

The above object is achieved in accordance with present invention by a method for fixing a thermoplastic cardiac lead tubing to a cardiac lead electrode or pin connector provided with outer thermoplastic fixation means, the method including the steps of
a) positioning an end portion of the tubing coaxially around a portion of the fixation means;
b) heating at least a section of the end portion of the tubing and at least a portion of the fixation means, so as to achieve fusing of said heated section of the end portion of the tubing to said heated portion of the fixation means; and
c) allowing the resulting fused portion to solidify.

This method results in a cardiac lead according to the first aspect of the present invention. The heating of the thermoplastic tubing and fixation means material to a temperature close to the melting points thereof causes fusing of the materials. The fused materials form a “seamless” connection when allowed to solidify.

The heating can be performed by any conventional means and preferably by conductive, irradiative or ultrasonic heating. More preferably the heating and fusing are performed by heat welding, providing a fast and reliable fixation of the tubing to the electrical contact means. Most preferably, the heat is applied locally to said section of the end portion of the tubing and to the fixation means, so as to avoid possibly negative influence on other parts of the cardiac lead. For example, the distal end of a cardiac lead may comprise a steroid plug, which is sensible to heat. Irradiative heating may be performed by laser. Ultrasonic heating may be performed as ultrasonic welding.

Supplementary heat treatment, such as annealing (e.g. to avoid environmental stress cracking), may additionally be performed.

The design of said electrode or pin connector is preferably as defined in relation to the first aspect of the invention.

In both aspects of the invention, the material of the thermoplastic tubing, as well as the material of the thermoplastic fixation means, preferably comprises a thermoplastic polymer. Such a material allows for heating to cause melting and for fusing, respectively. Property-wise such materials should preferably be rigid or flexible thermoplastic materials suitable for extrusion or injection molding and be biocompatible. Common materials fulfilling these requirements are polyurethanes, polyolefins (such as UHMWPE, HDPE, LDPE, etc.), polycarbonates (plexiglass), polyesters (such as PET, Dacron), polyamides (such as Nylon), polyimides, etc., the common characteristics being that the materials can be melted, reformed while in the molten state, and then become solid again in the new form upon cooling.

Generally, to facilitate fusing and to allow a durable joint, the melting point, melt index and/or hardness of the thermoplastic polymer of the two materials should preferably be close to each other. Furthermore, both materials should preferably be free from impurities. In particular, moisture and soot (resulting from e.g. insufficient cleaning of equipment used) should be avoided.

With regard to the above-mentioned general guidelines for the choice of materials and for fusing of thermoplastic polymers, preferred materials are characterized by having soft and hard segments in the polymer backbone, joined by reaction of isocyanates. Hence, any type of modified thermoplastic polyurethane material, such as a polyether polyurethane, a polycarbonate polyurethane, a poly(siloxane-carbonate) polyurethane, or a poly(ether-siloxane) polyurethane, may preferably be used for either of the thermoplastic tubing or the thermoplastic fixation means.

Depending on the general properties required for the tubing, such as resistance to abrasion, flexibility, durability and surface properties, a preferred thermoplastic polymer is a polyether polyurethane comprising soft segments and hard segments or a poly(ether-siloxane) polyurethane comprising soft segments and hard segments. In a polyether polyurethane, the soft segments may comprise polytetramethyleneoxide (PTMO) (such as in Pellethane™, Dow Chemical Company) or polyhexamethyleneoxide (PHMO). A preferred polyether polyurethane is a Pellethane™, more preferred is Pellethane™ 2363-55D. In a poly(ether-siloxane) polyurethane, the soft segments may comprise polyhexamethyleneoxide (PHMO) and polydimethylsiloxane (PDMS) (such as in Elast-Eon™). A preferred poly(ether-siloxane) polyurethane is an Elast-Eon™, more preferred is Elast-Eon™ 2A. The hard segments of the polyether polyurethane or the poly(ether-siloxane) polyurethane, respectively, may comprise be a diisocyanate, such as methylenediphenyldiisocyanate (MDI), and a diol, such as butanediol (BDO), as a chain extender.

Depending on the properties required for the fixations means, such as compatibility with the tubing, castability, melting point etc., a preferred thermoplastic polymer is a polyether polyurethane comprising soft segments and hard segments. The soft segments may comprise polytetramethylenedioxide (PTMO) (such as in Tecothane®, Thermedics Polymer Products) or polyhexamethylenedioxide (PHMO). The hard segments may be a diisocyanate, such as methylenediphenyldiisocyanate (MDI) and a diol, such as butanediol (BDO), as a chain extender. A preferred polyether polyurethane is a Tecothane®, more preferred is Tecothane® 1075D.

### DESCRIPTION OF THE DRAWINGS

FIGS. 1A and 1B respectively show cross-sectional views of the fixing of a thermoplastic tubing material to an electrode or a pin connector provided with outer thermoplastic fixation means by fusion.

### DESCRIPTION OF THE PREFERRED EMBODIMENTS

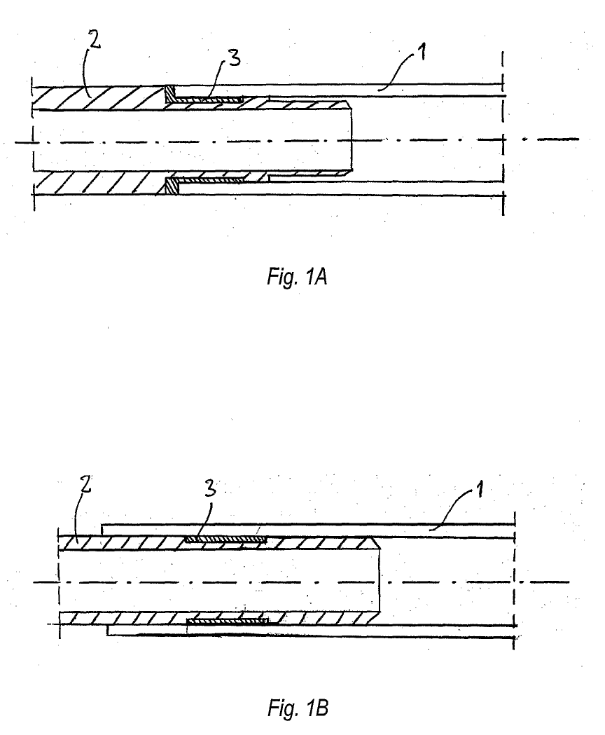

In a cardiac lead according to FIG. 1A, a tubing 1 was fixed to a ring electrode 2. The ring electrode had been provided with a fixation element 3. The tubing was fixed to the ring electrode by heat welding of the tubing to the fixation means, i.e. two semicircular jaws clamped and heated the arrangement of tubing and fixation means to obtain a joint.

Another embodiment of a tubing 1 fixed to an electrode 2 provided with a fixation element 3 is shown in FIG. 1B.

### Example

Tensile testing of cardiac leads according to the embodiment shown in FIG. 1A was performed according to EN45502-2-1 (CEN/CENELEC). The electrode was made of Pt/Ir 90/10 and was provided with a fixation element made of Tecothane® 1075D, which had been injection moulded onto the electrode. The fixation element of 0.1 mm thickness and 1.4 mm length was provided in a 0.1 mm recess of 1.4 mm length extending around the perimeter of the electrode (1.55 mm diameter).

Three tubing materials were tested:
1) Elast-Eon™ 2A, inner diameter 1.70±0.025 mm, outer diameter 1.88±0.025 mm;
2) Elast-Eon™ 2A, inner diameter 1.70±0.025 mm, outer diameter 1.95±0.025 mm; and
3) Pellethane™ 2363-55D, inner diameter 1.69±0.05 mm, outer diameter 2.14±0.05 mm.
   The heat welding was performed according to two different methods:

A) Two stage process including
welding at 135° C. for 5 s (jaws of unhardened tool steel) (fusion of tubing to fixation means) and—compression at 110° C. for 8 s (jaws of unhardened tool steel); and
B) Welding at 240° C. for 40 s (jaws of unhardened tool steel with a teflon surface).
   Each group tested comprised 8-13 samples. Pull force results (N) at break are presented as mean values and standard deviation in Table 1.

TABLE 1
Tensile testing, pull force

| Tubing | Heat | Mean | Standard |
|---|---|---|---|
| material | welding | value (N) | deviation |
| 1 | A | 10.8 | 1.9 |
| 2 | A | 9.0 | 0.8 |
| 2 | B | 9.5 | 2.4 |
| 3 | A | 14.4 | 2.3 |

Safe cardiac lead performance requires (EN45502-2-1 CEN/CENELEC)) the cardiac lead to withstand 5 N pull force during 1 minute. All samples complied with this requirement. Furthermore, the pull force mean value at fracture was ≧9 N. All samples present a small standard deviation, thus being suitable for accurate production.

Although modifications and changes may be suggested by those skilled in the art, it is the invention of the inventor to embody within the patent warranted heron all changes and modifications as reasonably and properly come within the scope of his contribution to the art.

We claim as our invention:

1-26. (canceled)

27. A cardiac lead comprising:
a thermoplastic tubing;
a termination element selected from the group consisting of an electrode and a pin connector;
a thermoplastic fixation element disposed on an exterior of said termination element; and
said thermoplastic tubing having an end portion coaxially surrounding a portion of said thermoplastic fixation element, with a section of said end portion of said tubing being thermoplastically fused to said fixation element, to fix said tubing to said termination element.

28. A cardiac lead as claimed in claim 27 wherein said termination element has a recess in which said thermoplastic fixation element is disposed.

29. A cardiac lead as claimed in claim 27 wherein said thermoplastic fixation element extends substantially completely around a perimeter of said termination element.

30. A cardiac lead as claimed in claim 27 wherein at least one of said thermoplastic fixation element and said thermoplastic tubing is formed of a material selected from the group consisting of polyurethanes, polyolefins, polyamides, and polyamides.

31. A cardiac lead as claimed in claim 30 wherein at least one of said thermoplastic fixation element and said thermoplastic tubing is formed of a material selected from the group consisting of polyether polyurethane, polycarbonate polyurethane, poly(siloxane-carbonate) polyurethane, and poly(ether-siloxane) polyurethane.

32. A cardiac lead as claimed in claim 27 wherein said thermoplastic tubing is comprised of a polyether polyurethane comprising soft segments and hard segments.

33. A cardiac lead as claimed in claim 32 wherein said soft segments are formed of a material selected from the group consisting of polytetramethyleneoxide and polyhexamethyleneoxide.

34. A cardiac lead as claimed in claim 32 wherein said hard segments comprise a diisocyanate and a diol.

35. A cardiac lead as claimed in claim 27 wherein said thermoplastic tubing is formed of poly(ether-siloxane) polyurethane comprising soft segments and hard segments.

36. A cardiac lead as claimed in claim 35 wherein said soft segments are formed of a material selected from the group consisting of polyhexamethyleneoxide and polydimethylenessiloxane.

37. A cardiac lead as claimed in claim 35 wherein said hard segments comprise a diisocyanate and a diol.

38. A cardiac lead as claimed in claim 27 wherein said thermoplastic fixation element is formed of polyether polyurethane comprising soft segments and hard segments.

39. A cardiac lead as claimed in claim 38 wherein said soft segments are formed of a material selected from the group consisting of polytetramethyleneoxide and polyhexamethyleneoxide.

40. A cardiac lead as claimed in claim 38 wherein said hard segments comprise a diisocyanate and a diol.

41. A method for fixing a thermoplastic tubing to a termination element selected from the group consisting of an electrode and a pin connector, said method comprising the steps of:
providing a thermoplastic fixation element disposed an exterior of said termination element;
positioning an end portion of said thermoplastic tubing coaxially around a portion of said thermoplastic fixation element;
heating at least a section of and at least a section of said end portion of said tubing to thermoplastically fuse said section to said fixation element; and
allowing said fused sections to solidify to fix said tubing to said termination element.

42. A method as claimed in claim 41 comprising heating said sections of said fixation element and said end portion of said tubing by a heating technique selected from the group consisting of conductive heating, radiant heating, and ultrasonic heating.

43. A method as claimed in claim 41 comprising heating said sections of said fixation element and said end portion of said tubing by heat welding.

44. A method as claimed in claim 41 comprising positioning said fixation element in a recess at said exterior of said termination element.

45. A method lead as claimed in claim 41 comprising forming said thermoplastic fixation element substantially completely around a perimeter of said termination element.

46. A method as claimed in claim 41 comprising forming at least one of said thermoplastic fixation element and said thermoplastic tubing of a material selected from the group consisting of polyurethanes, polyolefins, polyamides, and polyimides.

47. A method as claimed in claim 46 comprising forming at least one of said thermoplastic fixation element and said thermoplastic tubing is formed of a material selected from the group consisting of polyether polyurethane, polycarbonate polyurethane, poly(siloxane-carbonate) polyurethane, and poly(ether-siloxane) polyurethane.

48. A method as claimed in claim 41 comprising forming said thermoplastic tubing of a polyether polyurethane comprising soft segments and hard segments.

49. A method as claimed in claim 48 comprising forming said soft segments of a material selected from the group consisting of polytetramethyleneoxide and polyhexamethyleneoxide.

50. A method as claimed in claim 48 comprising forming said hard segments of a diisocyanate and a diol.

51. A method lead as claimed in claim 41 comprising forming 27 said thermoplastic tubing of poly(ether-siloxane) polyurethane comprising soft segments and hard segments.

52. A method lead as claimed in claim 51 comprising forming said soft segments of a material selected from the group consisting of polyhexamethyleneoxide and polydimethylenessiloxane.

53. A method lead as claimed in claim 51 comprising forming said hard segments of a diisocyanate and a diol.

54. A method as claimed in claim 41 comprising forming said thermoplastic fixation element of polyether polyurethane comprising soft segments and hard segments.

55. A method as claimed in claim 54 comprising forming said soft segments of a material selected from the group consisting of polytetramethyleneoxide and polyhexamethyleneoxide.

56. A method as claimed in claim 54 comprising forming said hard segments of a diisocyanate and a diol.
